(19) United States
(12) Patent Application Publication
(10) Pub. No.: US 2025/0196142 A1
(43) Pub. Date: Jun. 19, 2025

(54) DEVICES, METHODS, AND SYSTEMS FOR ENRICHING CELLULAR SAMPLES BY PHYSICAL PROPERTIES

(71) Applicant: Inso Biosciences, Inc., Ithaca, NY (US)

(72) Inventors: Adam Bisogni, Ithaca, NY (US); Harold Craighead, Ithaca, NY (US); Harvey Tian, Ithaca, NY (US); Mohamed Zakarya Rashed, Ithaca, NY (US); Irem Bastuzel, Ithaca, NY (US); Azade Tahmasebi, Ithaca, NY (US)

(21) Appl. No.: 18/847,435

(22) PCT Filed: Mar. 16, 2023
(86) PCT No.: PCT/US2023/064504

§ 371 Date: Sep. 16, 2024

Related U.S. Application Data
(60) Provisional application No. 63/320,391, filed on Mar. 16, 2022.

(51) Int. Cl. B01L 3/00; C12Q 1/6883

(52) U.S. Cl. CPC B01L 3/502761; C12Q 1/6883; B01L 2200/025; B01L 2200/028; B01L 2200/0647; B01L 2400/0487; B01L 2400/086; C12Q 2600/156

(57) ABSTRACT

Disclosed are microfluidic flow-based devices and methods for enriching cellular samples by physical properties. This includes the use of devices for separating cells and particles based on physical size or mechanical properties in a planar microfluidic format. This improves the value and quality of the DNA sequence data derived from these samples. An exemplary method includes enriching the DNA content from pathogenic or other microorganisms in a biological sample by removing the more prevalent and unwanted DNA from cells of the host organism.

### BACKGROUND

Separating different cell types from a heterogenous sample is critical process for certain types of biological analyses. As one example, removing host-cells, host-cell genomic DNA (gDNA) and other contaminants from biological samples is a major hurdle when processing metagenomic samples for effective and efficient down-stream analysis of microbial populations. When DNA is extracted from a biological sample that is expected to contain a microbial community, most of the DNA in the sample is actually derived from the host organism, and not the microbes of interest. Therefore, downstream analysis such as genetic sequencing end up generating data primarily sourced from the host genome, which is uninformative of the microbial community and therefore undesirable. As a result, only a small amount of data is produced from the population of interest—the microbial community. This process renders metagenomic samples extremely expensive, ineffective, insensitive, and impractical for use in clinical settings.

Resolving this issue is critical to leverage the full power of metagenomic samples in clinical settings because sequencing data of such samples can provide critical guidance for personalized care to treat infections, identifying infections of unknown origins, enhancing diagnoses, or for generating epidemiological data for monitoring infectious disease outbreaks.

Therefore, there is a need in the art to further develop methods for separating and enriching cell populations from biological samples based on physical properties.

### SUMMARY

This invention relates to methods for enriching certain cell populations from a biological sample based on physical properties. Additionally, large fragments of acellular gDNA may also be removed from a sample based on size. A facile method is enabled whereby the sample is flowed through the channel, resulting in the entrapment and therefore removal of the cells depending on their physical size, while allowing for smaller sized cells to flow through unimpeded. The resulting processed sample is enriched for the cells of interest with a greatly reduced amount of the unwanted cells and any acellular gDNA present in the sample. The desirable cells that flow to the output of the chip may then be processed using any downstream analytical platform, including but not limited to genetic sequencing.

As some embodiments of the workflow, a biological sample containing a heterogenous mix of cells with different diameters or shapes is loaded into the microfluidic chip via direct syringe loading or through a standard pressure driven fluid control system. The flow rate can be defined by a fluid control system, or controlled manually via a syringe. The rate at which the solution is allowed to flow through the device is flexible to meet the needs of the user's sample volume and time requirements. In some embodiments of the device, the length of the microfeature array consists of defined distances between each microfeature which gradually decrease. As such, when the solution flows through the array, larger particles such as host-cells become trapped by the decreasing size between microfeatures. The spacing size and overall design can vary in different embodiments to meet the needs of different sample and cell types, but it never becomes smaller than the sizes of the smallest cells of interest, ensuring that they will pass through the array without difficulty and can be recovered from the output reservoir.

In certain aspects, the disclosure provides a microfluidic device for isolating a cell or a particle from a sample. The device includes a support having an inlet port for receiving the sample, an outlet port for dispensing the flow-through, and a microfluidic channel disposed within the support and extending from the inlet port to the outlet port. The microfluidic channel includes at least a first array of microfeatures that capture the cell or particle. The microfeatures may be micropillars, squares, triangles, rectangles, inverse/negative outline triangles, cross shapes, hexagonals, diamonds, or any combination thereof. The microfeatures may be separated from each other by spacings between 2 micrometers and 150 micrometers. The array may include at least two sub-arrays that have (a) different microfeatures and/or (b) different microfeature spacing sizes. The at least 2 sub-arrays may be arranged in sequence along the channel in the direction of flow. Preferably, a first sub-array closer to the input port has microfeature spacing size that is larger than microfeature spacing size of a second sub-array that is closer to the output port. In that arrangement, the first sub-array captures large particles or cells, while other parts of the sample flow through towards the outlet and the second subarray captures smaller particles or cells. For example, the second sub-array may specifically trap bacteria (e.g., after the first subarray captures larger plant or animal cells). The second subarray may capture other small particles such as nuclei, sperm cells, or any other particle of interest. In certain embodiments, the 2 sub-arrays of microfeatures have different microfeature spacing sizes, in which (a) the sub-array closest to the input port comprises microfeatures that are separated by 50 micrometers; and (b) the sub-array closest to the output port comprises microfeatures that are separated by 5 micrometers. Additionally or alternatively, the array of microfeatures may have spacing between the microfeatures that becomes smaller along the array in the direction from input to output. The array may include at least 2 different microfeature spacing sizes. The array may include at least 3, 4, or 5 different microfeature spacing sizes. Preferably, each sub array or each region along the array will capture a cell or particle of a different size than the preceding subarray or region along the array.

### BRIEF DESCRIPTION OF FIGURES

FIG. 1. Overview of workflow for utilizing an exemplary system for enriching a microbial community from a metagenomic sample. In this example, the sample contains a mixture of human cells, human acellular DNA, and microbial cells.
(1) User starts with a biological sample containing undesirable human cells, human acellular gDNA and the desired microbial cells.
(2) An example of microfluidic device prior to loading sample. Features including an input port for loading the solution, an array of microfeatures spaced at defined distances, and an output port or reservoir for collection of processed sample.
(3) An example of microfluidic device after flowing the sample through. The larger human cells and acellular human gDNA are trapped within the microfeature array, unable to progress to the output port, while the smaller microbial cells can traverse to the output port unimpeded.
(4) Recovered processed sample is now enriched for the microbial population.

FIG. 2 shows a first embodiment of microfeature geometries within an array as small dense micropillars.

FIG. 3 shows an array with micropillars larger and more spaced apart than in the first embodiment, specifically with microfeatures of circular shape; micropillars.

FIG. 4 shows microfeatures of square or rectangular shape.

FIG. 5 shows microfeatures of triangular shape.

FIG. 6 shows microfeatures of inverse/negative outline triangles.

FIG. 7 shows microfeatures of rotated square shapes (e.g., rotated about 45 degrees relative to a top-to-bottom flow direction).

FIG. 8 shows microfeatures of cross shapes.

FIG. 9 through FIG. 13 show exemplary anti-clogging designs at the border of two different microfeature spacings for cell separation. Direction of flow is indicated by input port and output port markings.

FIG. 9 shows a microfeature array having two microfeature spacing sizes. Flow runs from top to bottom. Light shade indicates larger microfeature spacings, while darker shading indicates smaller microfeature spacings. Larger cells would therefore become stuck at this interface while smaller cells would pass through the array. Due to the increased surface area of the interface with this design, the risk of clogging of the channel is mitigated.

FIG. 10 shows a first variation of “square wave” interface design.

FIG. 11 shows a second variation of “square wave” interface design, demonstrating that the height of the square can be changed to meet various design needs.

FIG. 12 shows a first (symmetrical) saw-tooth variation of the interface design.

FIG. 12 shows a second (skewed) saw-tooth variation of the interface design.

FIG. 14 through FIG. 17 show design examples demonstrating how two different microfeature gradient interfaces and designs are stacked to produce multi-stage filtration. Direction of flow is indicated by input port and output port markings.

FIG. 14 shows a device that provides two stages of filtration, a tall square wave interface followed by a shorter square wave interface.

FIG. 15 shows a device that provides two stages of filtration, first a tall square wave interface, and then a saw-tooth interface.

FIG. 16 shows a device that provides two stages of filtration, first a broad saw-tooth interface, and then a saw-tooth with an increased frequency interface.

FIG. 17 through FIG. 19 show design examples of how multistage filtration interfaces can be spaced out at various lengths along the array. Direction of flow is indicated by input port and output port markings.

FIG. 17 shows a short length between microfeature interfaces of spacing sizes.

FIG. 18 shows an intermediate length between microfeature interfaces of spacing sizes.

FIG. 19 shows an extended length between microfeature interfaces of spacing sizes.

FIG. 20 is micrograph of a human epithelial cells from a saliva sample trapped within the microfeature array, demonstrating the ability of the microfeatures to capture the larger cells while letting smaller particles through. Direction of flow is indicated by arrow.

FIG. 21 is a fluorescent micrograph showing human acellular gDNA that was loaded into the channel trapped within the pillar array, unable to progress to the output port and therefore filtered from the solution. Direction of flow is indicated by arrow.

FIG. 22 shows quantification of the filtration of 15 micrometer polystyrene beads from a solution after flowing through the microfeature array. Smallest feature spacing size: 9 micrometers.

FIG. 23 shows quantification of the recovery of 5 micrometer polystyrene beads from a solution after flowing through the microfeature array. Smallest feature spacing size: 9 micrometers.

FIG. 24 is a photomicrograph of a device with a sawtooth boundary.

FIG. 25 shows a device with first and second arrays of microfeatures that meet at a sawtooth boundary.

FIG. 26 shows the device of FIG. 25 in use with phosphate buffered saline (PBS).

FIG. 27 shows the device of FIG. 25 after use after PBS has washed through.

FIG. 28 shows a device with sawtooth boundaries and walls on the sawtooth peaks.

FIG. 29 shows the device of FIG. 28 after washing with PBS.

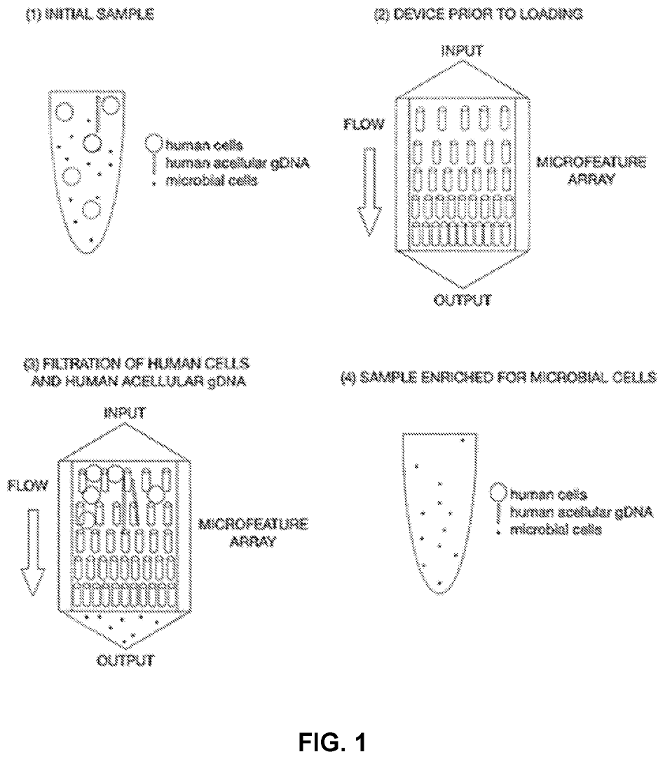

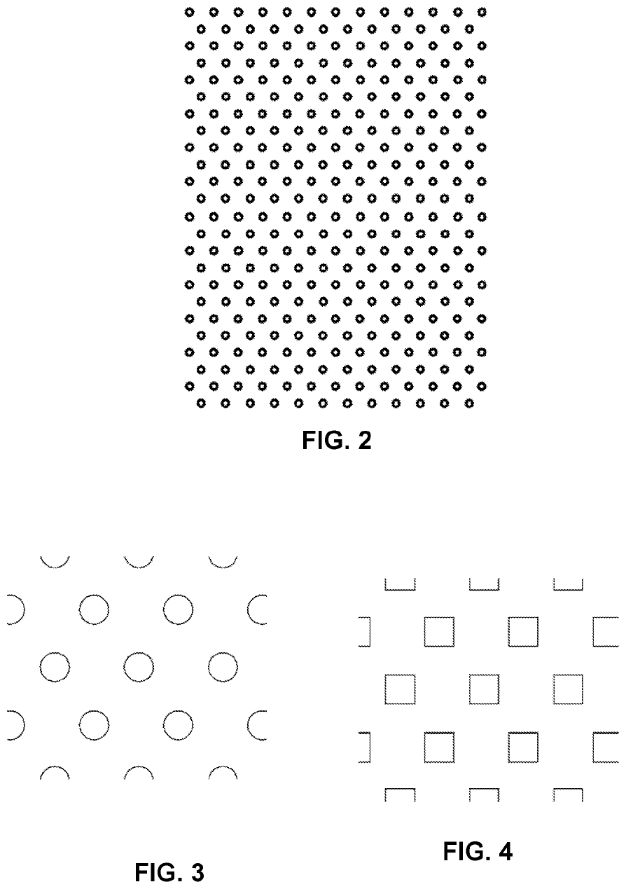

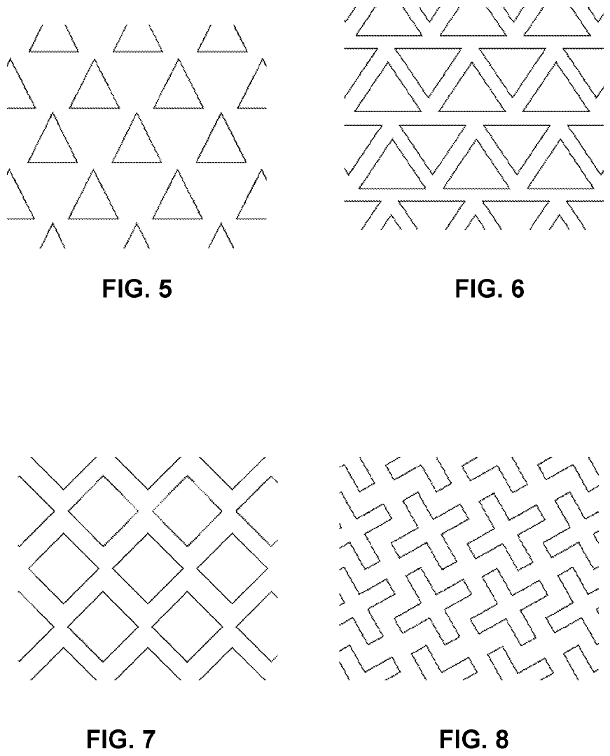

### DETAILED DESCRIPTION

The present disclosure relates to methods for enriching microbial populations from a biological sample by removing the physically larger host-cells and acellular host-cell gDNA. Some of the underlying developments of the present disclosure are supported by the following patents, patent applications, and publications: U.S. Pat. No. 10,947,528, Craighead et al.

Microfluidic device for extracting, isolating, and analyzing DNA from cells; U.S. Pat. No. 9,926,552, Craighead et al. Microfluidic device for extracting, isolating, and analyzing DNA from cells; US Patent Application Publication No. US 2007/0259424 A1; Chinese Patent Application Publication No. CN102212458A; International Patent Application Publication No. WO2019010788A1; US Patent Application Publication No. US20130143197A1; Benitez, J. J., Topolancik, J., Tian, H. C., Wallin, C. B., Latulippe, D. R., Szeto, K., Murphy, P. J., Cipriany, B. R., Levy, S. L., Soloway, P. D., et al. (2012). Microfluidic extraction, stretching and analysis of human chromosomal DNA from single cells. Lab on a Chip 12, 4848; Tian, H. C., Benitez, J. J., and Craighead, H. G. (2018). Single cell on-chip whole genome amplification via micropillar arrays for reduced amplification bias. PLOS ONE 13, e0191520; Reinholt, S. & Craighead, H. G. (2018) Microfluidic Device for Aptamer-Based Cancer Cell Capture and Genetic Mutation Detection. Anal Chem. 90 2601-2608. doi: 10. 1021/acs.analchem.7b04120; H. W. Hou et al. Microfluidic Devices for Blood Fractionation. Micromachines 2011, 2(3), 3 19-343, World Wide Web at doi.org/10.3390/mi2030319. Each of these is incorporated by reference herein in their entirety.

Disclosed are microfluidic flow-based devices and methods for enriching a desired cell population based on size in a biological sample by removing the larger unwanted cells. This includes the use of devices for separating cells and particles based on physical size or mechanical properties in a planar microfluidic format. In some embodiments, a biological sample containing a mixed cell population (e.g., host cells and microbial cells, where the microbial cells are smaller in diameter than the host cells) can be applied to a microfluidic device with features that allow only the microbial cells to pass through the device. As the cells flow through the device, the larger host cells become restricted and trapped within appropriately spaced microstructures (such as micropillars) incorporated in the microfluidic device, while the smaller microbial cells flow through the device unimpeded. Long strands of gDNA derived from the host cell that may be free in the sample solution may also be trapped by the microstructures. Thus, the microbial sample is enriched by the removal of host-cells and any acellular genomic DNA (gDNA) from the host organism. This improves the value and quality of the DNA sequence data derived from these samples.

### Definitions

The articles “a” and “an” are used herein to refer to one or to more than one (i.e., to at least one) of the grammatical object of the article. By way of example, “an element” means one element or more than one element.

As used herein, the term “sample” will be understood to encompass any fluid, solution or mixture, either isolated or detected as a constituent of a more complex mixture, or synthesized from precursor species. The term “sample” encompasses any biologically derived sample or biological sample, including but not limited to blood, plasma, serum, lymph, saliva, tears, cerebrospinal fluid, urine, sweat, plant or vegetable extracts, semen, in vitro cell culture, tissue homogenates (e.g., animal tissue homogenate, plant tissue homogenate, etc.), and ascites fluid. The sample may or may not be native. For example, the sample may comprise acellular species, e.g., broken cells, genomic DNA

The term “microbe” is art-recognized, and includes microorganisms such as bacteria, fungus, algae, and virus.

The term “particle” is art-recognized and includes any particle that can pass through or captured by the device of the present disclosure. As used herein, the particle includes acellular species, such as nucleus, nuclei, organelles, genomic DNA, and metabolites such as starch and polyphenols, large biological molecules including proteins and lipids and sugars, inorganic (human-made or natural) matter, as well as clumps or conglomerates of those foregoing materials.

### System

This invention is based on methods for utilizing a micropillar array in a microfluidic channel to enrich small particles or cells by physically trapping larger cells or particles and gDNA that are present in the solution to remove them from the rest of the sample. The enrichment therefore occurs via passive removal of the unwanted larger cells and gDNA as they become stuck within the spacings in the array and are inhibited from progressing through the channel. FIG. 1 outlines a typical workflow of a disclosed system.

### Microfeature Array

### Methods for Fabricating the Microfluidic Device Comprising Microfeatures

Methods for fabricating the microfluidic device employ standard approaches of lithography and etching to create molds that can be replicated in Polydimethylsiloxane, (PDMS) or other polymers using molding, embossing or injection molding. An example for a method to fabricate the device is described in Benitez et al. The molded PDMS can be easily bonded to a glass plate to complete the structure, or the device may be constructed by a combination of different polymeric materials.

### Microfluidic Device Comprising Microfeatures

Various types of microfeatures may be used, such as pillars (e.g., micropillars), squares, triangles, rectangles, inverse/negative outline triangles, cross shapes, diamonds, hexagonals, other geometries. In certain embodiments, the microfluidic device comprises an array of microfeatures comprising a combination of various types of microfeatures.

In some embodiments, the array comprises micropillars. In some embodiments, the array does not comprise micropillars. In some embodiments, the array comprises a combination of micropillars and at least one other type of microfeatures.

In some embodiments, the spacing between the microfeatures becomes smaller along the array (i.e., in the direction from input to output; in the direction of the flow, e.g., FIG. 1).

The combination of channel dimensions, microfeature sizes, microfeature spacing, and the organization of the microfeature array can be varied for a desired type of sample or sample volume. For example, smaller microfeature spacings would be used to trap smaller cells or cell nuclei while larger spacings would be used to select larger cells from a mixture containing smaller entities in the sample.

In some aspects, microfluidic flow-based devices for enrichment of a particular cell population based on size comprises of a microfluidic channel having an inlet port and an outlet port to allow a flow in a direction from the inlet port toward the outlet port; and an array of microfeatures disposed within the microfluidic channel.

In some embodiments, the microfeatures have diameters or areas between 3 micrometers and 15 micrometers, but could range from 1 micrometer to more than 100 micrometers (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 micrometers; and any range between these values). In some embodiments, the microfeatures (e.g., micropillars) have diameters between 6 micrometers and 40 micrometers.

In some embodiments, the microfeatures have a height between 15 and 30 micrometers, but could range from 5 to 300 micrometers (e.g. 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 120, 140, 160, 180, 200, 220, 240, 260, 280, or 300 micrometers; and any range between these values).

In some embodiments, the microfeatures are separated from each other by spacings between 2 micrometers and 150 micrometers, but could range from 0.1 micrometers up to more than 300 micrometers (e.g., 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 micrometers; and any range between these values).

In some embodiments, an important feature is that the spacing between the microfeatures becomes smaller along the array to enable filtration of larger cells from the smaller ones.

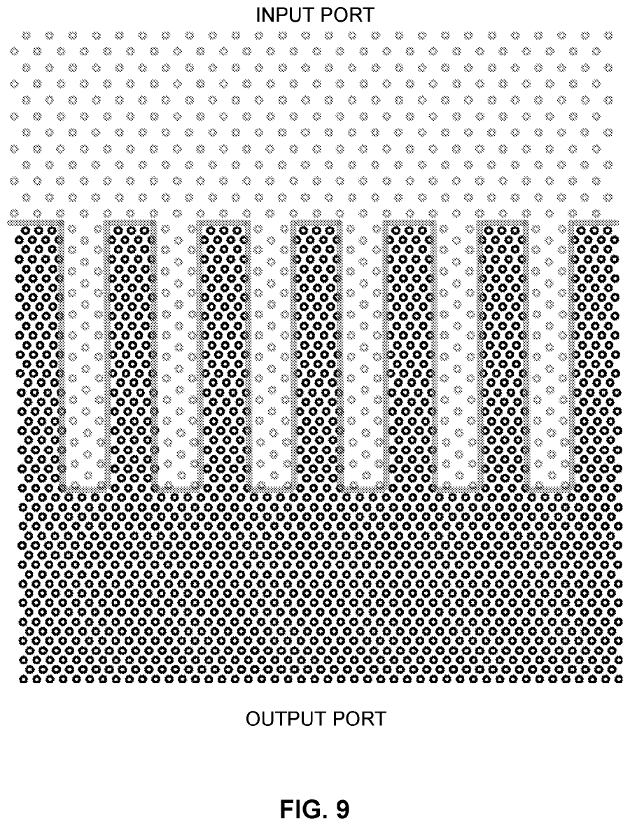

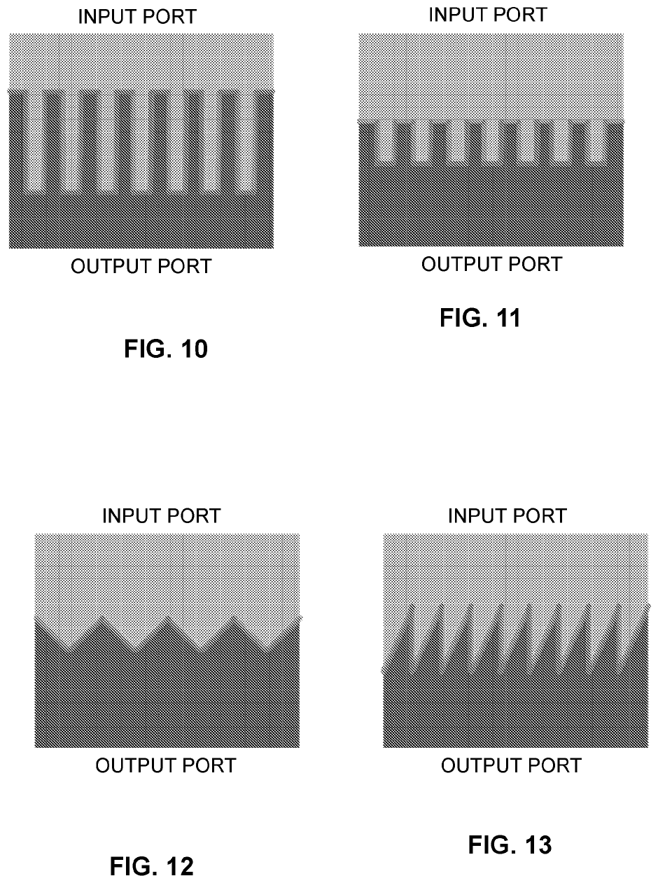

In some embodiments, the array of microfeatures comprises at least 2 different microfeature spacing sizes (e.g., FIG. 9 through FIG. 13). FIG. 9 through FIG. 13 show arrays that include at least 2 different microfeature spacing sizes.

In some embodiments, the array comprises at least 3, 4, or 5 different microfeature spacing sizes.

In certain embodiments comprising two microfeature spacing sizes (one interface between two microfeature spacing sizes), the microfeatures on the slide closest to the input port are spaced 50 micrometers apart, and the microfeatures in region closest to the output port are spaced by 5 micrometers.

In certain embodiments comprising three microfeature spacings sizes (two interfaces between regions of different spacings), the region closest to the input port has microfeature spacings of 50 micrometers, the region in the middle of the array has microfeature spacings of 25 micrometers, and the region closest to the output port has microfeature spacings of 5 micrometers.

In certain embodiments comprising four microfeature spacing sizes (three interfaces between regions of different spacings), the region closest to the input port has microfeature spacings of 150 micrometers, the next region has microfeature spacings of 50 micrometers, the second to last region has spacings of 20 micrometers, and the final region closest to the output port has spacing of 5 micrometers.

In some embodiments, the array is bounded within an area of 1 square millimeter or greater (e.g., 1, 1.11.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 100 square millimeters; and any range between these values); and the microfeatures are arranged within the array in a defined configuration.

In some embodiments, the microfeatures comprise a polymer (e.g., they are made of a polymer). In some embodiments, the micropillars are fabricated using a lithographic process. In some embodiments, the device further comprises one or more canals extending to within said area of the array of micropillars. In some embodiments, the one or more canals are aligned along the flow direction. In some embodiments, said diameters, said spacings, said area, said configuration, or a combination thereof is selected based on type of said cell or cell nucleus.

### Designs for Microfeature Geometries and Arrangements

In some embodiments, the array length comprises a simple gradient of microfeatures separated by defined spaces along the length of the array.

In some embodiments, the borders between some changes in spacing of microfeatures may take a shape (e.g., square wave) to create an interface between two different microfeature spacing sizes with an increased surface area. This is intended to allow for the capture of certain cell sizes while minimizing the risk of any clogging that may reduce the flow of the smaller sized cells through the array.

In some embodiments, the arrays consist of smaller “sub arrays” that are staggered with respect to each neighboring “sub array” such that the gradients are offset within one channel to reduce the probability of clogging.

In some embodiments, the microfeature spacing gradient is repeated one or more times along the length of a channel to produce a “multi-filter” stage effect.

In some embodiments, the microfeature geometries are altered to produce localized regions of higher flow (e.g., microfeatures arranged in square lattice) or reduced flow (e.g., microfeatures arranged in an offset diamond lattice).

In some embodiments, a combination of the aforementioned arrangements, geometries, and designs are used.

For example, in certain embodiments, the array comprises at least 2 sub-arrays, wherein the at least 2 sub-arrays comprise (a) different microfeatures; and/or (b) different microfeature spacing sizes.

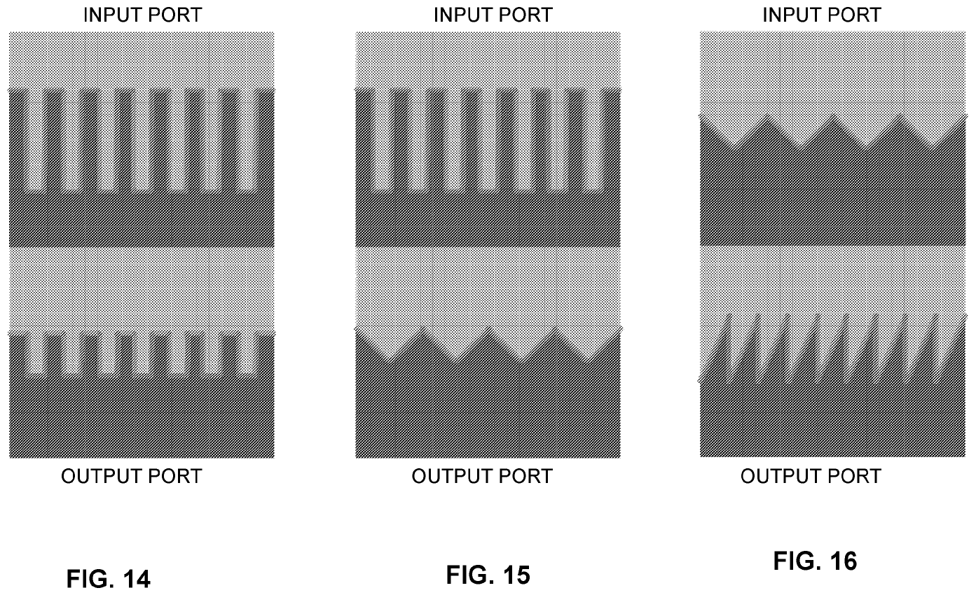

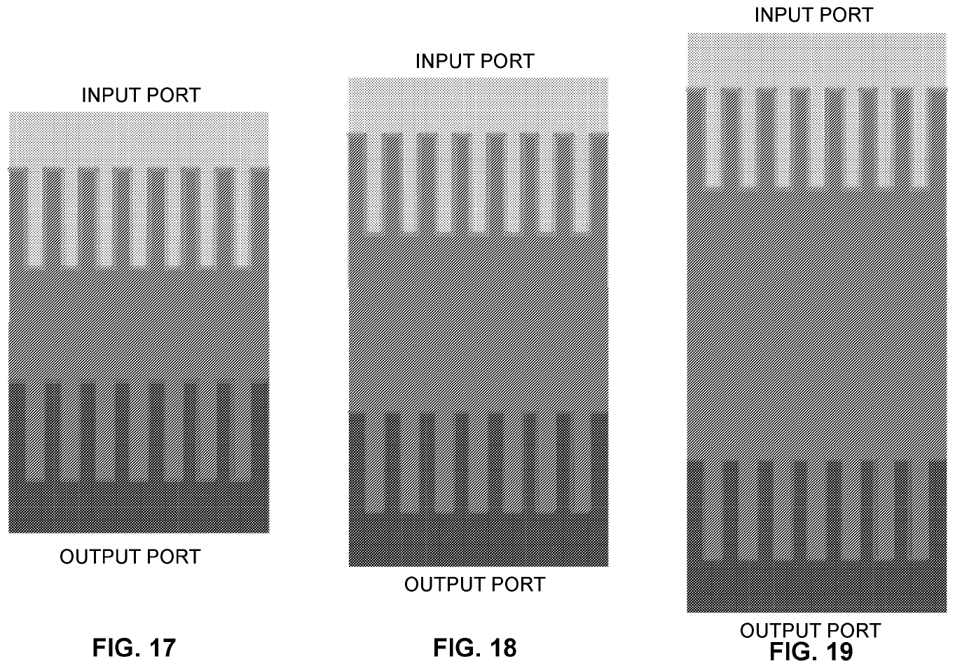

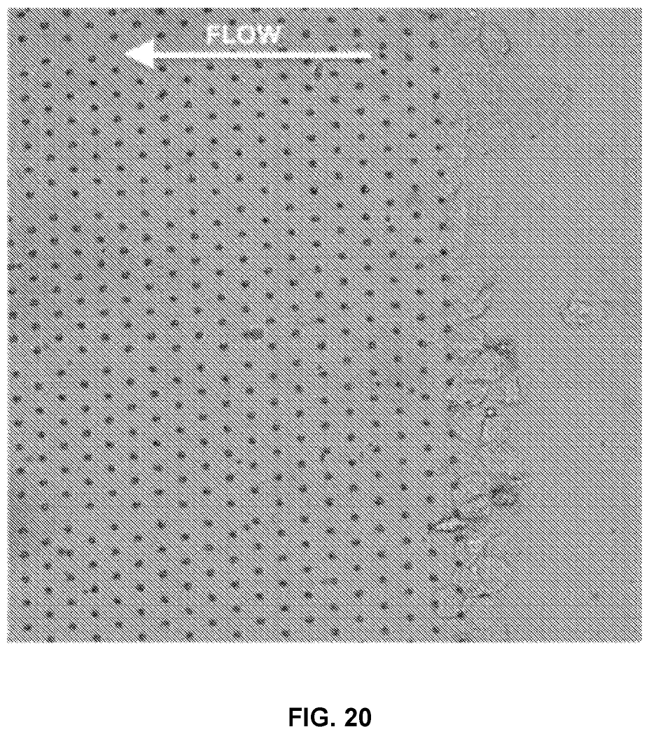

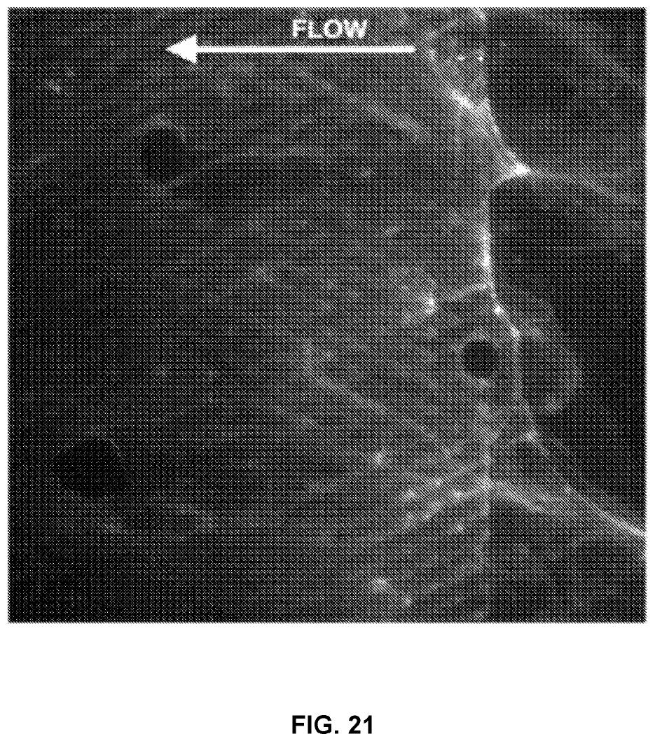

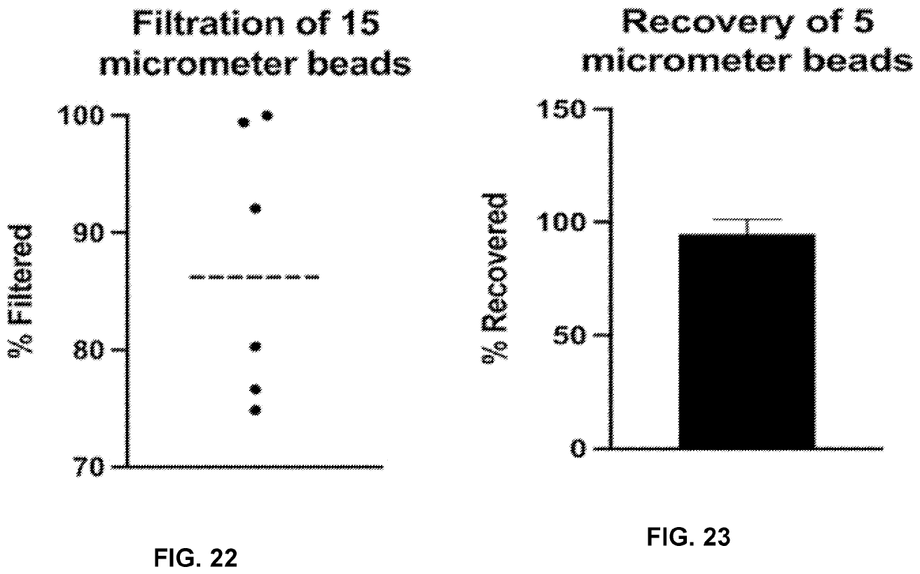

In some embodiments, the at least 2 sub-arrays are arranged in sequence along the channel parallel to the flow (see e.g., FIG. 9 through FIG. 19).

In some embodiments, the at least 2 subarrays are at least 3, 4, 5, or 6 sub-arrays.

In some embodiments, the at least 2 sub-arrays with different microfeature spacing sizes are arranged in sequence along the channel parallel to the flow (see e.g., FIG. 9 through FIG. 19). FIG. 9 through FIG. 19 show devices that include at least 2 sub-arrays arranged in sequence along the channel parallel to the flow.

In some embodiments, the sub-array closer to the input port comprises the microfeature spacing size that is larger than the microfeature spacing size of the sub-array that is closer to the output port.

In some embodiments, the microfeatures entrap, by size exclusion, non-target cells/particles and/or acellular genomic DNA

### Methods

In certain aspects, the present disclosure encompasses devices and methods of isolating cells/particles by entrapping non-target cells/particles within the device and allowing the target cells/particles to flow through.

In other aspects, the present disclosure encompasses devices and methods of isolating cells/particles by entrapping the target cells/particles within the device and allowing the non-target cells/particles to flow through.

A person of ordinary skill in the art would readily know, based on the present disclosure and the physical properties (size and/or shape) of the target cells/particles, how to adjust the spacing between the microfeatures to isolate the cells/particles according to aspects of the device.

### Loading and Processing of Samples in the Device

As shown in FIG. 1, the method begins by introducing the sample (e.g., whole blood, saliva, urine, respiratory secretions, cerebrospinal fluid) into the input port of the device, either through direct interface with a syringe or a fluidic control system. The fluid sample is the pushed through the microfluidic channel. The volume of the sample can range from 5 uL to I 00+mL. The rate at which the sample is pushed through the channel can be defined by the needs of the user and sample type.

### Recovery of Enriched Sample

To recover the enriched processed sample, the solution is collected from the output port reservoir. In other embodiments, tubing can be connected to the output port to direct the flow directly into a tube or other fluid container.

### Blocking Electrostatic Interactions

In some cases, due to electrostatic forces, smaller particles exhibiting negative surface charges on their may adhere to microfeatures in the array. To reduce this, a blocking agent (e.g., BSA, ionic detergent, or another similarly charged agent) may be used. The device may be coated with a blocking agent. For example, surfaces of the microfeatures on one or more of the arrays within the device and surfaces along microchannels may be coated. In another example, a blocking agent may be spiked into the sample prior to loading to reduce the electrostatic interaction between the PDMS microfeatures and the smaller particles from occurring.

### EXAMPLES

### Example 1 Fabrication of a Microfluidic Device

To fabricate the master molds, Microposit S1813 photoresist (Shipley; Marlborough, MA) is spun on silicon on insulator (SOI) wafers (Ultrasil; Hayward, CA) and exposed by UV contact lithography (EVG620, EVG Group; Albany, NY). The exposed resist is developed in 726MIF developer (Microchemicals) and the pattern is transferred into the 20 μm-thick top silicon layer by Bosch process in a Unaxis SLR 770 deep reactive ion etching system (Unaxis USA Inc.; St. Petersburg, FL). A monolayer of (1H, 1H, 2H, 2H Perfluorooctyl) Trichlorosilane is deposited on the etched wafers in a MVD100 molecular wafer deposition system (Applied Microstructures; San Jose, CA) to prevent sticking of the PDMS to the mold. Sylgard 184 (Dow Corning; Midland, MI) PDMS base resin is mixed with the curing agent at a 10:1 ratio, degassed under vacuum at room temperature, poured onto the master, and cured for 45 min at 150° C. The elastomer casting is then peeled off the mold and access holes to the input and outputs of the microchannels are created with a 1.5 mm biopsy punch (Sklar Instruments; West Chester, PA). To complete channel fabrication, the patterned PDMS is treated with oxygen plasma for 1 min and bonded to a 170-μm thick fused silica wafer (Mark Optics; Santa Ana, CA).

### Example 2 Isolating Microbial Cells Using a Microfluidic Device from Urine Samples

Using the microfluidic device prepared according to Example 1, a volume (possible range: 10 uL to over 50 mL) of urine from a patient suspected to have a urinary tract infection (bacteriuria) is injected into the microfluidic device via syringe loading or through a fluidic control system. The goal is to sequence the DNA of the bacteria present in the sample in order to determine the best antibiotic treatment to give the patient. However, the sequencing is severely limited by the contamination of host DNA and host cells (in this case, leukocytes or renal epithelial cells containing DNA) in the sample whose abundance of DNA greatly outweighs that of the bacterial DNA of interest. This greatly reduces the usefulness of the sequencing data because only a small amount of the sequencing reads will end up being from the bacteria species. As the leukocytes present in urine during bacteriuria are typically −8-25 micrometers in diameter and renal epithelial cells are typically −20-35 micrometers in diameter, the unwanted cells are much larger than bacteria present in the urine (which are typically less than 5 micrometers in size, with a variable shape). Thus, as the urine sample is flowed through the microfluidic device and the cells traverse through the array of microfeatures at defined spacing, the bacteria cells are isolated from the leukocytes. In this sample type, the effective final size cut-off could be −7 micrometers.

### Example 3 Isolating Microbial Cells from Whole Blood Samples

Using the microfluidic device prepared according to Example 1, a volume (possible range: 10 uL to over 50 mL) of whole blood (treated with or without anticoagulants such as ethylenediaminetetraacetic acid) is injected into the microfluidic device via syringe loading or through a fluidic control system. In this example, clinicians treating a patient with suspected sepsis need to quickly identify the bacteria. Typically sequencing of all the cells in blood would produce an overwhelming amount of sequencing reads generated from the host leukocytes and very little reads from the bacteria of interest.

To effectively identify the bacteria or other microbes through sequencing, the microbial cells must be isolated form the host cells and the host gDNA so that the host DNA is not carried over to the sequencing reaction where it will overtake the sequence reaction and ineffectively generate data on the microbes.

In a suspected patient with an infection, the whole blood will contain leukocytes (containing DNA, which typically range from −8 to 25 micrometers in diameter), erythrocytes (not containing DNA, which typically are ˜7 micrometers in diameter), and any bacteria, virus, or other microbial cells present (typically 0.5 to 5 micrometers in size, shape is variable). In the first filtration stage, all cells (leukocytes) greater than 7 micrometers are impeded by microfeatures. In a possible second filtrations stage, erythrocytes are trapped at a 5-micrometer stage to further clean up the sample.

Thus, as the fluid is pushed through an array of microfluidic geometries at defined spacings, only the smaller microbial cells would be recovered, thus enriching them.

### Example 4 Isolating Olfactory Sensory Neurons from Mouse Olfactory Epithelium Tissue

Isolation of certain cell types from a complex mixture of cells originating from a particular tissue type or organ is often desirable for research purposes. For example, in the study of olfactory sensory neurons (OSNs), it is difficult to obtain a pure, enriched population of OSNs due to the complex mixture of other cell types within the tissue. Typically, the OSNs are the smallest cell size in diameter (−10 micrometers), as the other cell types such as the basal cells and supporting cell types are much greater. To utilize the microfluidic device to enrich OSNs from a dissociated olfactory epithelium sample, the volume is flowed across a microfeature array with the smallest microfeature spacing size just over 10 micrometers. This enables the OSNs to flow to the output port, while capturing and therefore separating out all the other cell types.

### Example 5 Removal of Plant Compounds or Secondary Metabolites from a Crude Nuclei Plant Preparation

When sequencing plant DNA, nuclei must first be prepared from the plant tissue. A method to release plant nuclei is often used that crudely releases the nuclei from the cell walls but also contains unwanted plant compounds including secondary metabolites such as starch and polyphenols. These particles are difficult to remove and are unwanted as they inhibit sequencing reactions.

The crude samples are loaded into the microfluidic device that has microfeatures that will catch the nuclei but allow for the much smaller plant compounds or secondary metabolites such as starch or polyphenols to escape the array. Due to the small particle size of these compounds, a final microfeature size of −5 micrometers is used for effective separation. The plant nuclei are thus isolated/entrapped within the array and purified from the unwanted plant compounds.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

### Example 6 Forensic Sample Processing

Methods and devices of the disclosure are useful for processing samples including those collected using a forensic sample collection kit such as a rape test kit sometimes also referred to as a rape kit, sexual assault kit (SAK), a sexual assault forensic evidence kit (SAFE), sexual assault evidence collection kit (SAECK), sexual offense evidence collection kit (SOEC) and physical evidence recovery kit (PERK). A challenge in processing such forensic samples is in isolating essential evidence from other components of the sample. For example, such a forensic sample may have sperm cells intermingled with epithelial and other cells. Methods of the invention include isolating sperm cells from a complex sample by processing the sample through a device of the disclosure. A first array of microfeatures feature array will capture epithelial cells and allow sperm cells to pass through. A second an array of microfeatures will capture the sperm cells and allow other components of the sample (cell-free nucleic acids, fragments of cells walls, serum, water or saline or other solutions used in processing, etc.) to pass through and elute from the device. The then isolated sperm cells are available for analysis. For example, nucleic acid may be extracted from the sperm cells by a suitable method including, for example, using methods and devices shown in U.S. Pat. Nos. 9,926,552 and 11,602,747, both incorporated by reference.

### Example 7 Sawtooth Boundary Between Arrays of Pillars, No Walls

Embodiments of devices of the disclosure were constructed and used for isolating cells and particles from samples by processing the samples through the devices that were made.

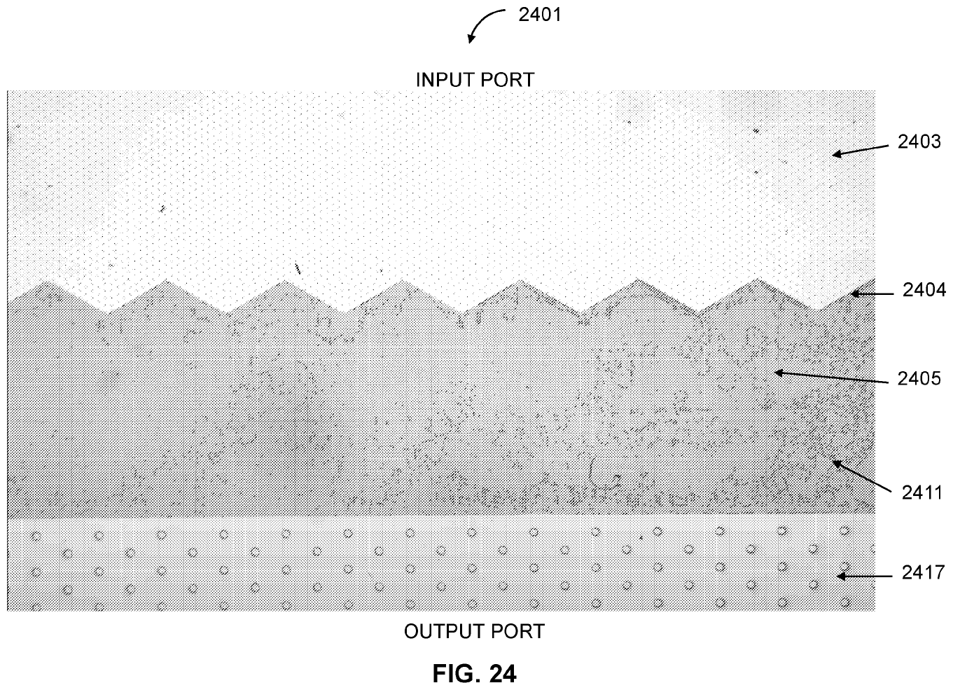

FIG. 24 is a photograph of a device 2401 with a sawtooth design. The device 2401 is useful for isolating a cell or a particle from a sample. The device 2401 includes a support 2417 having an inlet port for receiving the sample, an outlet port for dispensing the flow-through, and a microfluidic channel disposed within the support and extending from the inlet port to the outlet port. The microfluidic channel includes a first array of microfeatures 2403 and a second array of microfeatures 2405.

In the reproduced photomicrograph, the first array of microfeatures 2403 are micropillars that are visible (as dot-like marks in the picture). The second array of microfeatures 2405 includes very fine micropillars that are small enough and close enough together that they appear as a uniform gray color across the middle of the figures. The first array of microfeatures 2403 meets the second array of microfeatures 2405 along a saw-tooth shaped boundary 2404. There is no wall or other structure at the boundary. The boundary 2404 is simply the span across the microchannel at which a fluid sample passes from the first array of microfeatures 2403 to the second array of microfeatures 2405.

The device 2401 was manufactured from PDMS and the PDMS included some manufacturing imperfections 2411 that are visible as some irregularly spaced dark marks in the photomicrograph but the imperfections 2411 (dark marks) are not part of any array of microfeatures. The PDMS device 2401 includes a surrounding supporting structure 2417 that appears to include large pillars or columns (visible as about 70 circles in the bottom 10% of the photomicrograph). Those parts of the supporting structure 2417 hold the device 2401 together with appropriate dimensions for sample processing but do not participate directly in sample processing.

This depicted embodiment of the device 2401 was manufactured and used and found to work well for separating small particles (e.g., bacterial cells, sperm cells, nuclei, etc.) from complex samples (e.g., including larger cells). Just as with other devices of the disclosure, larger cells or particles get trapped in the first array of microfeatures 2403 and smaller cells or particles are trapped in the second array of microfeatures 2405.

### Example 8 Sawtooth Boundary Between Arrays of Pillars, Walls Between Peaks

Embodiments of devices of the disclosure were constructed and used.

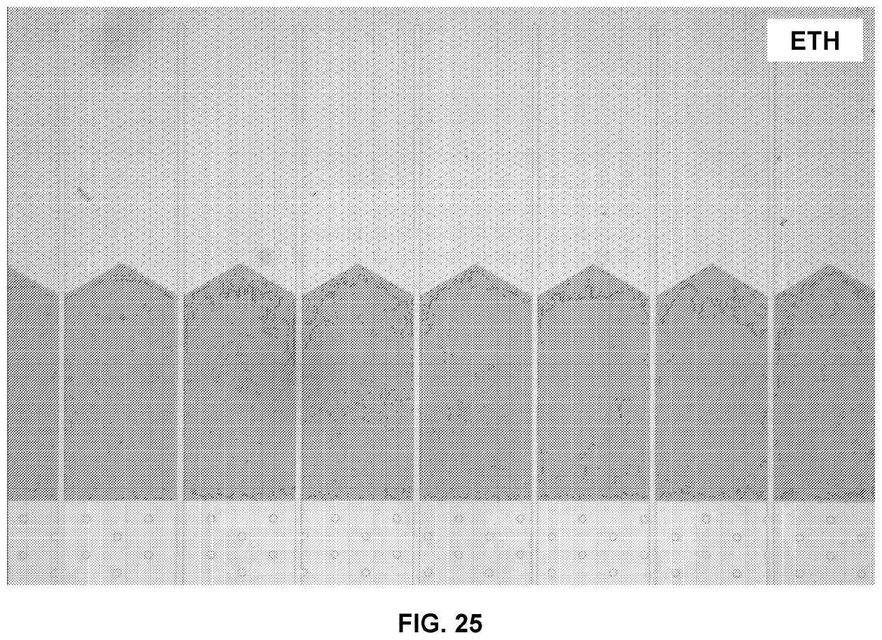

FIG. 25 shows an embodiment with first and second arrays of microfeatures in which the arrays meet at a sawtooth boundary (with some visible supporting structures at the bottom of the picture adjacent the output port). This embodiment of the device includes walls placed in the microchannel at the valleys of the sawtooth

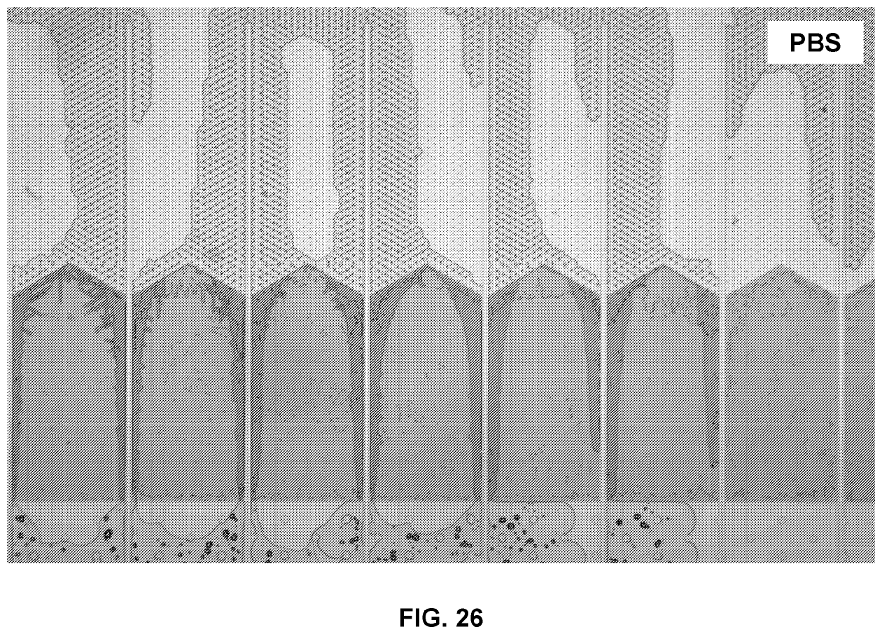

FIG. 26 shows the device of FIG. 25 in use with phosphate buffered saline (PBS).

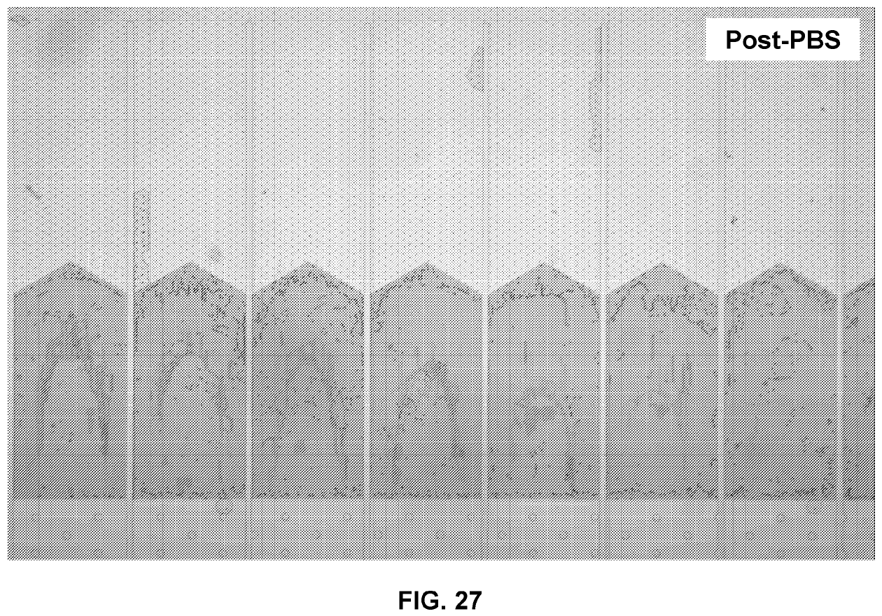

FIG. 27 shows the device of FIG. 25 after use after PBS has washed through. The depicted device with sawtooth boundary and walls between peaks was one of the devices according to the invention that was manufactured, tested, and found to work well for the disclosed methods.

### Example 9 Sawtooth Boundary Between Arrays of Pillars, Walls on Peaks

Embodiments of devices of the disclosure were constructed with sawtooth boundaries and walls on the sawtooth peaks and used in methods of the invention.

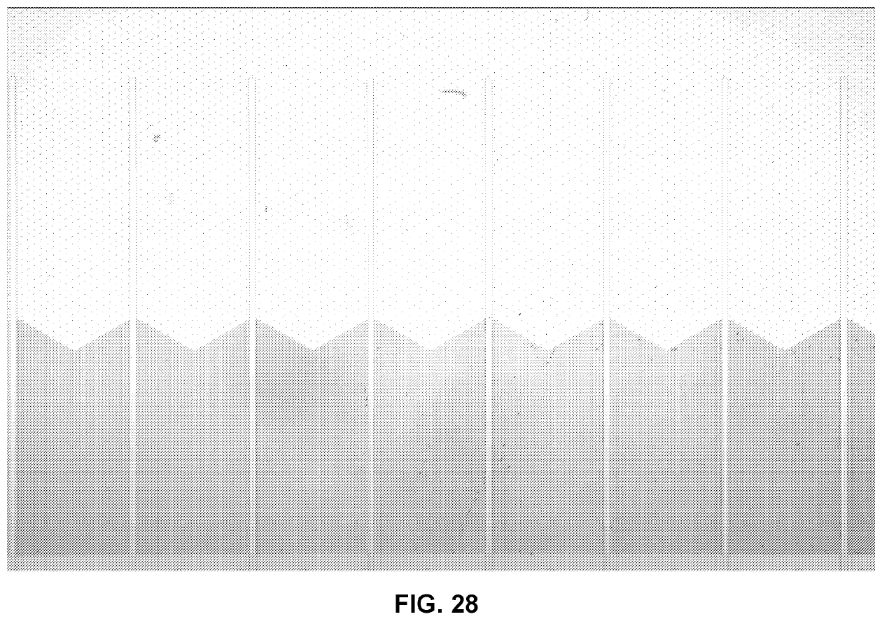

FIG. 28 shows a device with sawtooth boundaries between first and second arrays of microfeatures and walls on the sawtooth peaks.

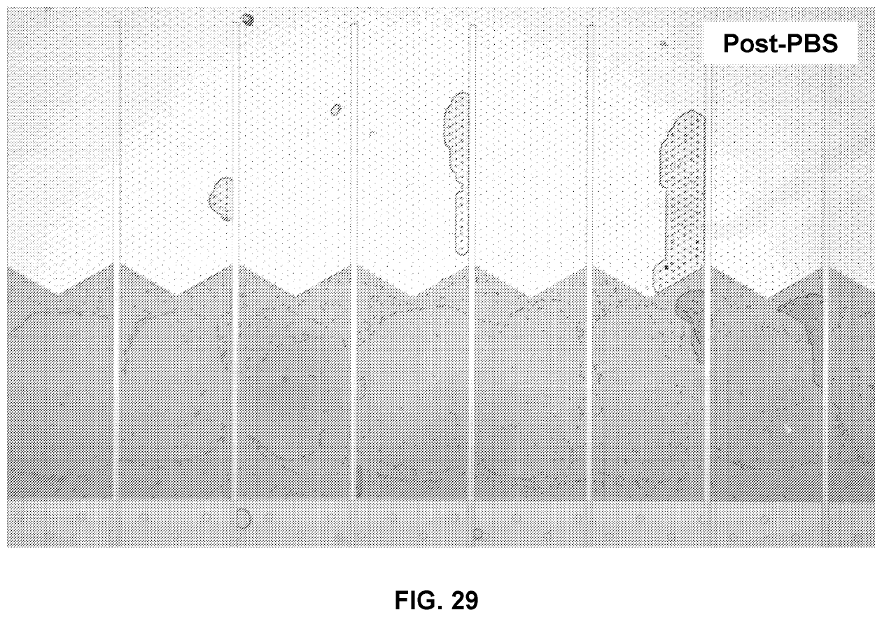

FIG. 29 shows the device of FIG. 28 after washing with PBS. Those devices that were manufactured and tested and shown to work well for methods of the invention tend to show that sawtooth boundaries between adjacent arrays of microfeatures work particularly well for separating particles from complex samples. Without being bound by any mechanism of action, it may be that the sawtooth boundary presents a sloped or ramped boundary, i.e., not perfectly perpendicular to direction of flow, and that clumps of material (cells, particles, biological molecules, etc.) are pushed by flow downslope in a manner that tends to break up the clumps and separate materials thereby avoiding clogging of the microchannel and promoting successful separation of the individual materials.

### INCORPORATION BY REFERENCE

All U.S. patents, and U.S. and PCT patent application publications mentioned herein are hereby incorporated by reference in their entirety as if each individual patent or patent application publication was specifically and individually indicated to be incorporated by reference. In case of conflict, the present application, including any definitions herein, will control.

### EQUIVALENTS

Those skilled in the art will recognize or be able to ascertain using no more than routine experimentation many equivalents to the specific embodiments of the present invention described herein. Such equivalents are intended to be encompassed by the following claims.

1. A microfluidic device for isolating a cell or a particle from a sample, the device comprising:
a support having an inlet port for receiving the sample, an outlet port for dispensing the flow-through, and a microfluidic channel disposed within the support and extending from the inlet port to the outlet port,
wherein the microfluidic channel comprises an array of microfeatures comprising micropillars, squares, triangles, rectangles, inverse/negative outline triangles, cross shapes, hexagonals, diamonds, or any combination thereof.

2-3. (canceled)

4. The device of claim 1, wherein the array comprises a combination of micropillars and at least one other type of microfeatures, wherein the micropillars have diameters between 3 micrometers and 30 micrometers.

5. (canceled)

6. The device of claim 1, wherein the spacing between the microfeatures becomes smaller along the array in the direction from inlet to outlet.

7-14. (canceled)

15. The device of claim 1, wherein the array comprises 3 sub-arrays of microfeatures comprising different microfeature spacing sizes, wherein:
(a) the sub-array closest to the inlet port comprises microfeatures that are separated by 50 micrometers;
(b) the sub-array in the middle comprises microfeatures that are separated by 25 micrometers; and
(c) the sub-array closest to the outlet port comprises microfeatures that are separated by 5 micrometers.

16. The device of claim 1, wherein the array comprises 4 sub-arrays of microfeatures comprising different microfeature spacing sizes, wherein:
(a) a first sub-array closest to the inlet port comprises microfeatures that are separated by 150 micrometers;
(b) a second sub-array comprises microfeatures that are separated by 50 micrometers;
(c) a third sub-array comprises microfeatures that are separated by 20 micrometers; and
(d) a fourth sub-array closest to the outlet port comprises microfeatures that are separated by 5 micrometers.

17. The device of claim 15, wherein the microfeatures have a diameter or area
(a) between 3 micrometers and 15 micrometers; or
(b) between 6 micrometers and 50 micrometers.

18. (canceled)

19. The device of claim 15, wherein the cell or the particle to be isolated is a microbe.

20. (canceled)

21. The device of claim 19, wherein the microfeatures entrap, by size exclusion, non-target cells/particles and/or acellular genomic DNA; and allow the target cells/particles to flow through.

22. The device of claim 1, wherein at least some microfeatures are separated from each other by spacing of less than or equal to 5 micrometers.

23-24. (canceled)

25. The device of claim 22, wherein the microfeatures entrap, by size exclusion, target cells; and allow non-target cells or particles to flow through.

26-29. (canceled)

30. A system comprising the device of claim 1, further comprising a fluid control module adapted to connect to the inlet port, wherein the fluid control module comprises a pressure-driven pump or a peristaltic pump.

31-32. (canceled)

33. The system of claim 30, further comprising one or more collection reservoirs adapted to connect to the outlet port, wherein the one or more collection reservoirs comprise a sample collection reservoir and a waste collection reservoir.

34. (canceled)

35. The system of claim 30, comprising a plurality of the arrays of micropillars within a plurality of the microfluidic channels, wherein the plurality of arrays has a respective plurality of inlet ports and/or a respective plurality of outlet ports and the plurality of inlet ports and/or the plurality of outlet ports are in an ordered configuration that aligns with a multi-well plate configuration.

36-39. (canceled)

40. A method of isolating a cell or a particle from a sample, comprising
processing the sample through a device comprising
a support having an inlet port for receiving the sample,
an outlet port for dispensing the flow-through, and
a microfluidic channel disposed within the support and extending from the inlet port to the outlet port, wherein the microfluidic channel comprises an array of microfeatures comprising micropillars, squares, triangles, rectangles, cross shapes, hexagons, diamonds, or a combination thereof; and
collecting the cell or the particle isolated via said processing.

41. The method of claim 40, further comprising:
collecting the sample devoid of the cell or the particle prepared via said processing.

42. The method of claim 40, further comprising analyzing nucleic acid sequences of the isolated cell or particle.

43. The method of claim 42, wherein the nucleic acid sequences of the isolated cell is analyzed by sequencing.

44. A method of diagnosing an infection or a disease in a subject, the method comprising analyzing the nucleic acid sequences according to the method of claim 42.

45. The method of claim 44, wherein the subject is a mammal, optionally wherein the subject is a human.

46. The method of claim 44, wherein the infection or the disease is a urinary tract infection or sepsis.
